(19) United States
(12) Patent Application Publication
(10) Pub. No.: US 2018/0153528 A1
(43) Pub. Date: Jun. 7, 2018

(54) COAXIAL BIOPSY NEEDLES

(71) Applicant: University of Florida Research Foundation, Inc., Gainesville, FL (US)

(72) Inventors: HITOMI GREENSLET, GAINESVILLE, FL (US); HAMIT KAHRAMAN, GAINESVILLE, FL (US)

(21) Appl. No.: 15/579,254

(22) PCT Filed: Jun. 1, 2016
(86) PCT No.: PCT/US16/35219

§ 371 Date: Dec. 4, 2017

Related U.S. Application Data
(60) Provisional application No. 62/170,928, filed on Jun. 4, 2015.

(51) Int. Cl. A61B 10/02

(52) U.S. Cl. CPC A61B 10/0275; A61B 10/0283

(57) ABSTRACT

Various examples of methods, devices, apparatus and systems are provided for coaxial biopsy needles. In one example, a coaxial biopsy needle includes an external needle including cutting edges at a distal end, the external needle affixed to an outer casing of a syringe at a proximal end and an inner stylette coaxially located within the external needle, where the inner stylette is affixed to a plunger of the syringe at a proximal end. In another example, a method includes inserting a coaxial biopsy needle into tissue to a first depth, maintaining the inner stylette at the first depth while further extending the external needle to a second depth in the tissue with the external needle cutting around a portion of the tissue surrounded by the external needle, and retracting the coaxial biopsy needle from the tissue to remove the portion of the tissue.

### CROSS REFERENCE TO RELATED APPLICATIONS

This application claims priority to, and the benefit of, co-pending U.S. provisional application entitled “COAXIAL BIOPSY NEEDLES” having Ser. No. 62/170,928, filed Jun. 4, 2015, which is hereby incorporated by reference in its entirety.

### STATEMENT REGARDING FEDERALLY SPONSORED RESEARCH OR DEVELOPMENT

This invention was made with government support under agreement CMMI-1266179 awarded by the National Science Foundation. The Government has certain rights in the invention.

### BACKGROUND

Needle biopsy procedures such as fine needle aspiration and core needle biopsy are used to extract tissue samples for diagnosis. Collection of larger samples allows for more accurate diagnosis of cancers. The combination of lower needle insertion force, less needle deflection, and reduced friction between the tissue and needle surface leads to a more effective biopsy procedure.

### SUMMARY

Embodiments of the present disclosure are related to coaxial biopsy needles and use thereof.

In one embodiment, among others, a coaxial biopsy needle comprises an external needle including cutting edges at a distal end, the external needle affixed to an outer casing of a syringe at a proximal end; and an inner stylette coaxially located within the external needle, the inner stylette affixed to a plunger of the syringe at a proximal end of the inner stylette. In one or more aspects of these embodiments, a distal end of the inner stylette can extend beyond the distal end of the external needle when the plunger is fully inserted into the syringe. The syringe can draw a vacuum between the inner stylette and the external needle when the distal end of the external needle is extended beyond the distal end of the inner stylette. The distal end of the stylette can taper to a point. The plunger can comprise a gasket configured to engage with an inner surface of the outer casing of the syringe thereby forming a vacuum tight seal.

In one or more aspects of these embodiments, the cutting edges can encircle the distal end of the external needle. The cutting edges can comprise an inner ridge extending inward from an inner surface of the external needle. In one or more aspects of these embodiments, the coaxial biopsy needle can comprise a clearance distance between an inner surface of the external needle and an outer surface of the inner stylette. The clearance distance can be in a range from about 0.025 mm to about 0.10 mm.

In another embodiment, a method comprises inserting a coaxial biopsy needle into tissue to a first depth, the coaxial biopsy needle comprising an inner stylette coaxially located within an external needle; maintaining the inner stylette at the first depth while further extending the external needle to a second depth in the tissue, the external needle cutting around a portion of the tissue surrounded by the external needle; and retracting the coaxial biopsy needle from the tissue to remove the portion of the tissue. In one or more aspects of these embodiments, a length of the portion of the tissue surrounded by the external needle can be substantially equal to a difference between the first depth and the second depth.

In one or more aspects of these embodiments, inserting the coaxial biopsy needle into the tissue can comprise inserting a distal end of the inner stylette and a distal end of the external needle into the tissue, where the distal end of the inner stylette is coaxially located within and extending beyond the distal end of the external needle. The distal end of the inner stylette can be maintained in a fixed position with respect to the external needle during insertion. The distal end of the inner stylette can be maintained in a fixed position with respect to the external needle while retracting the coaxial biopsy needle from the tissue. In one or more aspects of these embodiments, the method can comprise extracting the portion of the tissue from the outer needle by forcing the inner stylette through the outer needle.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A and 1B are graphical representations of biopsy sample methods in accordance with various embodiments of the present disclosure.

FIG. 2A illustrates an example of a coaxial biopsy needle in accordance with various embodiments of the present disclosure.

FIGS. 2B through 2D are images of an example of the coaxial biopsy needle of FIG. 2A in accordance with various embodiments of the present disclosure.

FIG. 3 is an example of obtaining a biopsy sample using the coaxial biopsy needle of FIG. 2A in accordance with various embodiments of the present disclosure.

FIG. 4A is an image of a test setup for testing sampling using the coaxial biopsy needles in accordance with various embodiments of the present disclosure.

FIGS. 4B and 4C are images illustrating the difference in biopsy sampling using the coaxial biopsy needle and fine needle aspiration, respectively, in accordance with various embodiments of the present disclosure.

FIG. 5 is an image of gelatin samples while drying in accordance with various embodiments of the present disclosure.

FIG. 6 includes bar graphs comparing test results of the coaxial biopsy needle in accordance with various embodiments of the present disclosure.

FIGS. 7A and 7B are images of gelatin biopsy samples obtained with different inner stylette diameters in accordance with various embodiments of the present disclosure.

### DETAILED DESCRIPTION

Disclosed herein are various embodiments related to coaxial biopsy needles, which can extract more tissue with less suction force and less tissue damage. The disclosed coaxial biopsy needle design can be used for both fine needle aspiration and core needle biopsy procedures. The needle tip geometry and the clearance between the inner stylette and the outer needle are factors affecting the biopsy performance, such as needle insertion force and amount of tissue extracted. Reference will now be made in detail to the description of the embodiments as illustrated in the drawings, wherein like reference numbers indicate like parts throughout the several views.

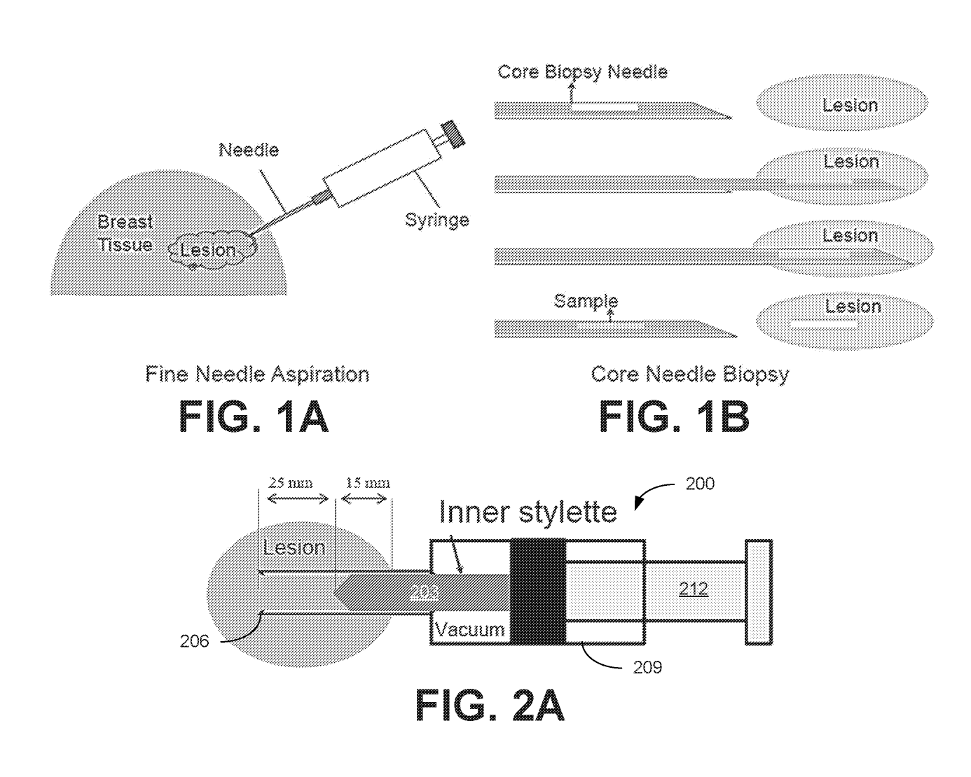

Referring to FIGS. 1A and 1B, shown are examples of biopsy needle sampling methods used to obtain tissue samples for the detection of breast cancer. FIG. 1A illustrates an example of a fine needle aspiration. As shown, a sample of the lesion can be extracted by a syringe via a fine needle. FIG. 1B illustrates an example of a core needle biopsy, where a sample is obtained in the side-opening trough of the biopsy needle. As shown, a core-extraction needle is initially inserted into a lesion with the outer sheath extended over the side-opening trough. When the outer sheath is retracted, the lesion is pressed into the side-opening trough. The outer sheath is then extended to obtain a sample of the lesion, which can then be removed from the core-extraction needle.

Challenges that exist in the development of a new biopsy needle design include improvements in cost effectiveness of the design, reduction in the discomfort experienced by the patient, and reduced damage to the extracted samples. A coaxial biopsy needle can be used as part of a cost effective biopsy method, which can result in less damage in the sampled tissue. The disclosed design can also extract a bigger sample while using a smaller needle diameter, which can reduce the discomfort felt by the patient during the biopsy.

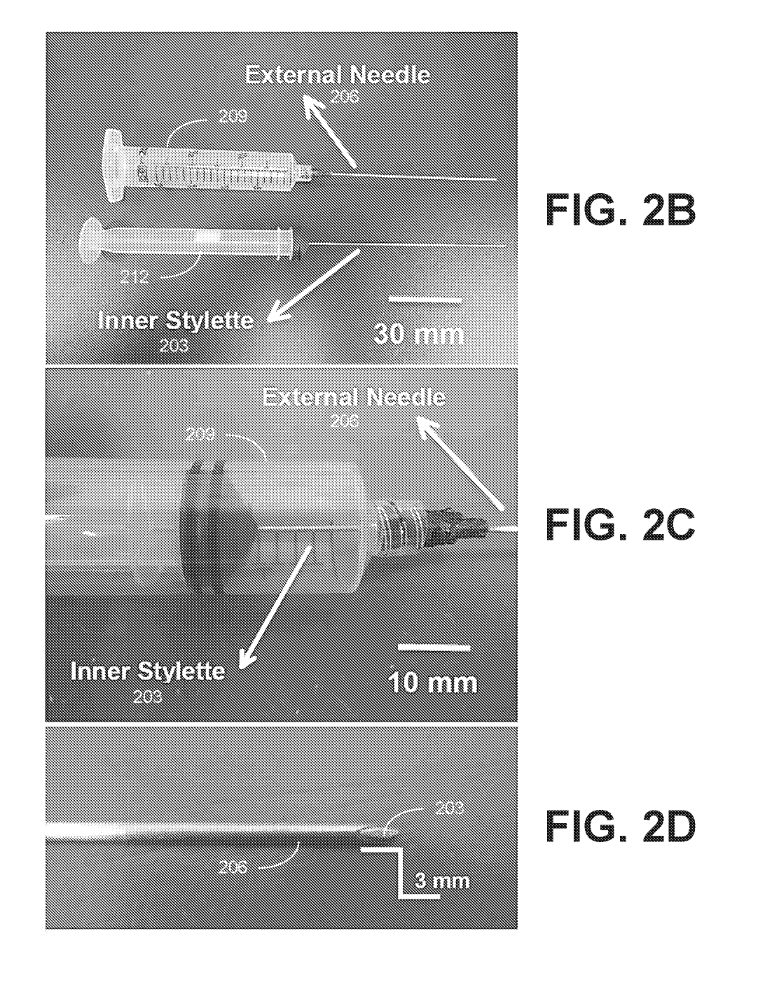

Referring to FIG. 2A, shown is an example of a coaxial biopsy needle 200 including an inner stylette 203 positioned within an external needle 206. The external needle 206 is affixed to an outer casing 209 of a syringe, and the inner stylette 203 is affixed to an end of a plunger 212, when inserted into the outer casing 209 substantially aligns the inner stylette 203 within the external needle 206. FIGS. 2B through 2C are images of an example of the coaxial biopsy needle 200. FIG. 2B illustrates the external needle 206 attached to the outer casing 209 of the syringe and the inner stylette 203 attached to the plunger 212. In the example of FIGS. 2B-2D, the external needle 206 is an 18 gauge (1.27 mm outer diameter, 1.14 mm inner diameter) needle with a length of 100 mm. The internal stylette is 127 mm long with a diameter of 0.94, 0.99, 1.04, or 1.09 mm. Other sizes (e.g., 16 gauge, 20 gauge, 22 guage, etc.) and lengths (e.g., 80 mm, 110 mm, 120 mm, 150 mm, etc.) can be used for the external needle 206 and for the corresponding inner stylette 203. FIG. 2C shows the coaxial biopsy needle 200 assembled with the inner stylette 203 inserted into the external needle 206. The clearance between an inner surface of the external needle 206 and an outer surface of the inner stylette 203 can be in a range from about 0.025 mm to about 0.10 mm or larger.

FIG. 2D shows the end of the inner stylette 203 extending beyond the end of the external needle 206. The inner stylette 206 is approximately 3 mm further from the lower tip of the external needle 203, and there is some clearance between inner stylette 206 and external needle 203. This clearance helps to transfer vacuum from the syringe to the needle tip during insertion and increase the efficiency of sampling by filling and holding more material in the needle 203. In addition, it helps to cut the sample tissue from the whole tissue.

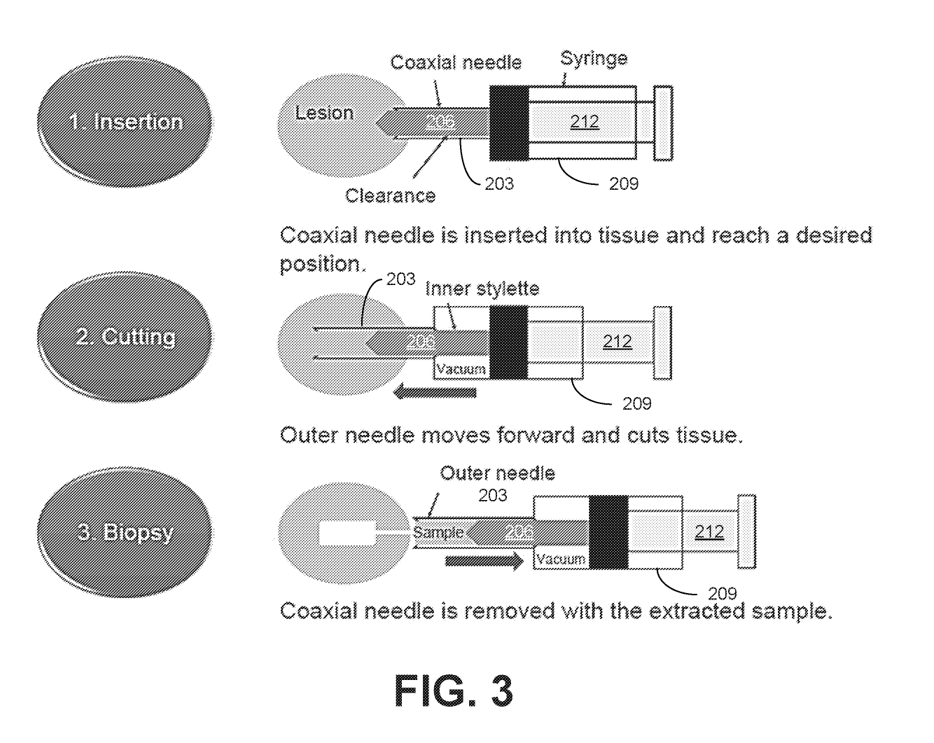

Referring to FIG. 3, shown is an example of acquiring a tissue sample using the coaxial biopsy needle 200. Initially, the coaxial biopsy needle 200 is inserted with the inner stylette 206 extending beyond the end of the external needle 203 until the desired position is reached (e.g., within a lesion). Next, the external needle 206 is extended into and cuts the tissue by moving the outer casing 209 forward while maintaining the position of the plunger 212. A vacuum is formed in the space between the outer casing 209 and the sealing of the plunger 212, which can help to draw the sample into the external needle 206. After extending the external needle 206 to the desired sample depth, the coaxial biopsy needle 200 is then removed from the tissue while maintaining the relative position of the outer casing 209 and plunger 212 of the syringe. In this way, a tissue sample is extracted within the external needle 206. The tissue sample can then be ejected from the coaxial biopsy needle 200 by moving the plunger 212 into the outer casing 209.

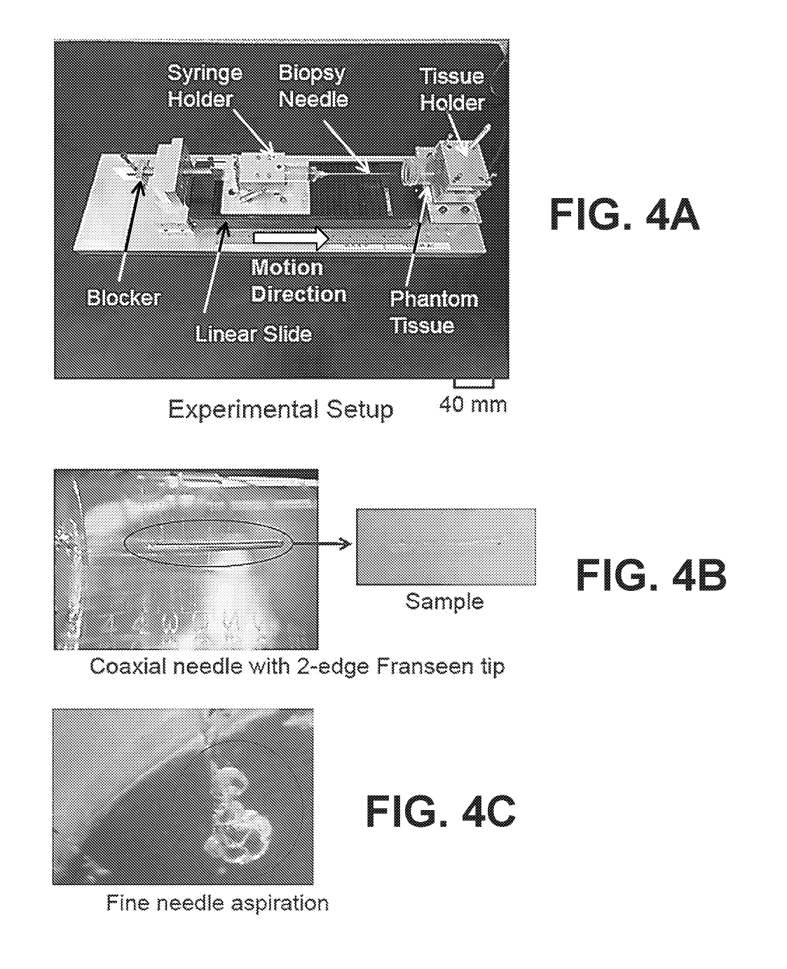

Biopsy sampling using a coaxial biopsy needle 200 was also carried out. FIG. 4A shows an image of a test setup used for biopsy sampling testing with a tissue phantom. The tissue phantom was made from 10 grams of gelatin mixed with 200 mL of water. FIG. 4B shows (1) an image of a sample being taken from the gelatin mixture with a coaxial biopsy needle 200 having a 2-edge Franseen tip and (2) an image of the extracted biopsy sample. When compared to the fine needle aspiration shown in FIG. 4C, the reduction in tissue damage using the coaxial biopsy needle 200 can be clearly seen.

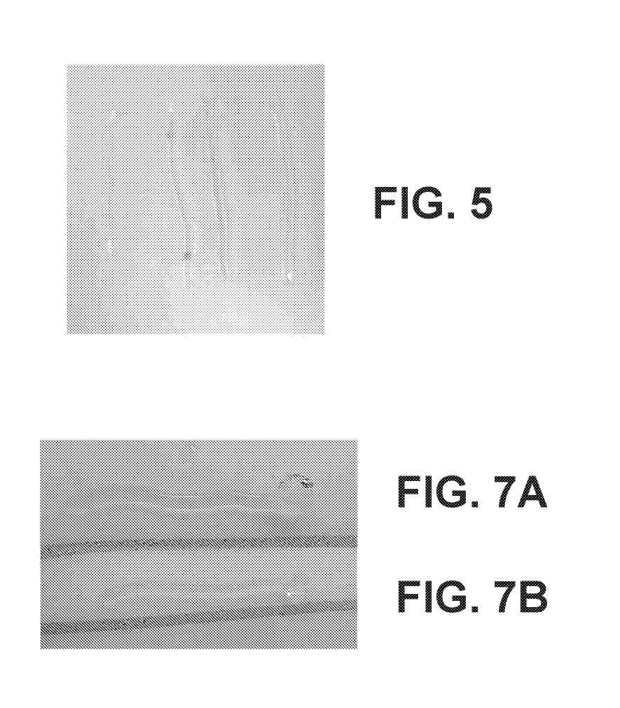

Experiments were run with 4 different inner stylettes 203 (FIG. 2A). Insertion velocities were chosen to be 1 mm/s and 5 mm/s. Biopsy samples were taken 5 times from different locations in the tissue phantom with the 1 mm/s insertion velocity and samples were taken 3-5 times from different locations with the 5 mm/s insertion velocity. Samples were taken after a 15 mm insertion from the surface of the tissue phantom and the external needle 206 was inserted 25 mm further as illustrated in FIG. 2A. Samples were evaluated for length and weight. The length was measured with a caliper, and weight was measured with a micro balance scale right after taking sample because the extracted gelatin could dry out and lose weight when exposed to air. FIG. 5 is an image showing gelatin biopsy samples during the drying process. The sample weights were measured under both wet and dry conditions.

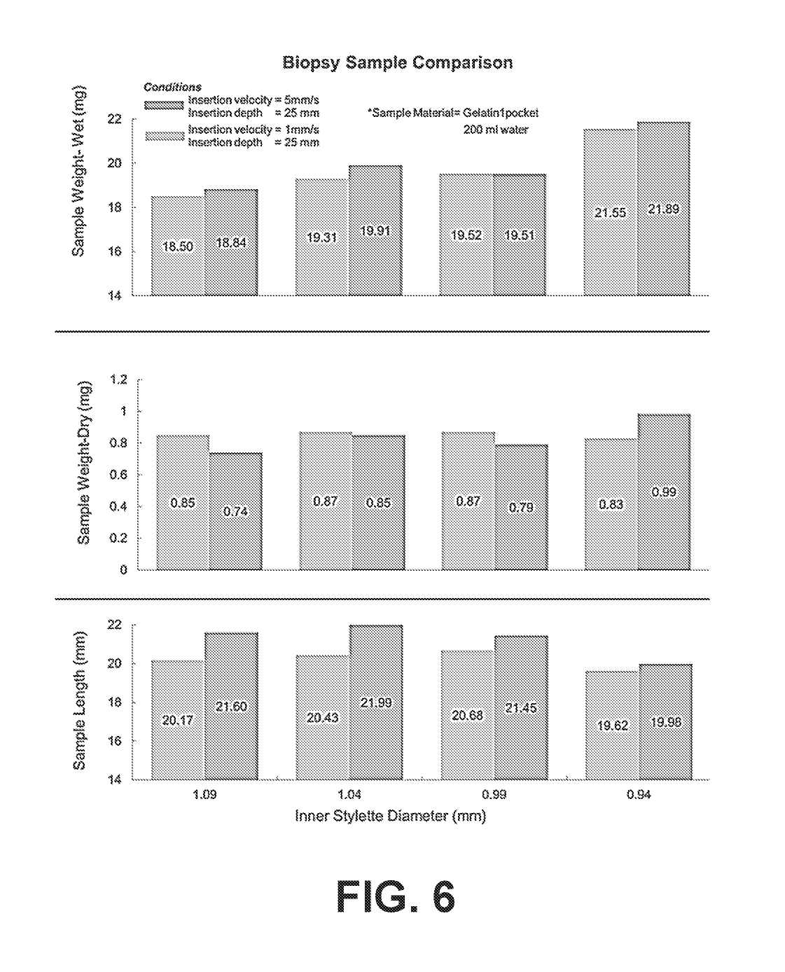

A comparison of the set of biopsy sample results is shown in FIG. 6. The peak weight was obtained with a 0.94 mm inner stylette 203 (FIG. 2A). This may be attributed to gelatin in the clearance because this gelatin mixture was easier to break apart and able to fill the clearance. The increase of the clearance between the inner stylette 203 and external needle 206 results in a ball shape at the tip of the sampled gelatin. FIGS. 7A and 7B are images of biopsy samples obtained with an inner stylette having diameters of 0.94 mm and 0.97 mm, respectively. The length shown in FIG. 7 was measured excluding the ball. Therefore, the length in the cases of stylette diameters of 0.97 and 0.94 mm decreased with increasing clearance. Increased insertion velocity was also found to increase the length of the acquired sample, but the sample weights appear to be almost the same. The higher the insertion velocity, the more the gelatin is stretched during cutting.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1. A coaxial biopsy needle, comprising:
an external needle including cutting edges at a distal end, the external needle affixed to an outer casing of a syringe at a proximal end; and
an inner stylette coaxially located within the external needle, the inner stylette affixed to a plunger of the syringe at a proximal end of the inner stylette.

2. The coaxial biopsy needle of claim 1, wherein a distal end of the inner stylette extends beyond the distal end of the external needle when the plunger is fully inserted into the syringe.

3. The coaxial biopsy needle of claim 2, wherein the syringe draws a vacuum between the inner stylette and the external needle when the distal end of the external needle is extended beyond the distal end of the inner stylette.

4. The coaxial biopsy needle of claim 2, wherein the distal end of the stylette tapers to a point.

5. The coaxial biopsy needle of claim 1, wherein the plunger comprises a gasket configured to engage with an inner surface of the outer casing of the syringe thereby forming a vacuum tight seal.

6. The coaxial biopsy needle of claim 1, wherein the cutting edges encircle the distal end of the external needle.

7. The coaxial biopsy needle of claim 6, wherein the cutting edges comprise an inner ridge extending inward from an inner surface of the external needle.

8. The coaxial biopsy needle of claim 1, comprising a clearance distance between an inner surface of the external needle and an outer surface of the inner stylette.

9. The coaxial biopsy needle of claim 8, wherein the clearance distance is in a range from about 0.025 mm to about 0.10 mm.

10. A method, comprising:
inserting a coaxial biopsy needle into tissue to a first depth, the coaxial biopsy needle comprising an inner stylette coaxially located within an external needle;
maintaining the inner stylette at the first depth while further extending the external needle to a second depth in the tissue, the external needle cutting around a portion of the tissue surrounded by the external needle; and
retracting the coaxial biopsy needle from the tissue to remove the portion of the tissue.

11. The method of claim 10, wherein a length of the portion of the tissue surrounded by the external needle is substantially equal to a difference between the first depth and the second depth.

12. The method of claim 10, wherein inserting the coaxial biopsy needle into the tissue comprises inserting a distal end of the inner stylette and a distal end of the external needle into the tissue, where the distal end of the inner stylette is coaxially located within and extending beyond the distal end of the external needle.

13. The method of claim 12, wherein the distal end of the inner stylette is maintained in a fixed position with respect to the external needle during insertion.

14. The method of claim 10, wherein the distal end of the inner stylette is maintained in a fixed position with respect to the external needle while retracting the coaxial biopsy needle from the tissue.

15. The method of claim 10, comprising extracting the portion of the tissue from the outer needle by forcing the inner stylette through the outer needle.
